(19) United States
(12) Patent Application Publication
(10) Pub. No.: US 2025/0099520 A1
(43) Pub. Date: Mar. 27, 2025

(54) Method for Synthesizing Selenium Nanoparticles from Edible Mushrooms

(71) Applicant: KING FAISAL UNIVERSITY, Al-Ahsa (SA)

(72) Inventors: HANY MOHAMED ABD EL-LATEEF AHMED, AL-AHSA (SA); MAI MOSTAFA KHALAF ALI, AL-AHSA (SA); MOHAMED SAYED YOUSSEF, Sohag (EG); IBRAHIM MOHAMED ABDELHALIM MOHAMED, Sohag (EG); SANAA IBRAHIM AHMED ABUDAHAB, Sohag (EG); MARWA MAHMOUD ABDEL-KAREEM, Sohag (EG)

(21) Appl. No.: 18/371,084

(22) Filed: Sep. 21, 2023

(51) Int. Cl. A61K 36/07; A61K 9/14; A61K 33/04; A61K 33/38; A61P 31/04; A61P 31/10; B82Y 5/00; C12P 3/00

(52) U.S. Cl. CPC A61K 36/07; A61K 9/14; A61K 33/04; A61K 33/38; A61P 31/04; A61P 31/10; B82Y 5/00; C12P 3/00

(57) ABSTRACT

A method of preparing selenium nanoparticles (NPs) using an edible mushroom extract. In an embodiment, the edible mushroom extract is derived from the species Pleurotus floridanus. In an embodiment, the method includes combining a mushroom extract with a source of selenium to provide an extract mixture, and maintaining the mixture at a pre-determined temperature until the selenium nanoparticles are formed. These nanoparticles can be used for antimicrobial purposes.

### BACKGROUND

### 1. Field

The disclosure of the present patent application relates to synthesis of selenium nanoparticles, and particularly, to synthesis of selenium nanoparticles using an extract of edible mushrooms.

### 2. Description of the Related Art

Recently, nanoparticles have demonstrated important uses in a variety of fields. Nanoparticles have been used in electronics, sensing, optics, and medicine, for example.

Synthesis of nanoparticles has been achieved by a variety of methods, including physicochemical, thermal decomposition, electrochemical, microwave assisted, sonochemical, solvothermal, photosynthesis, photochemical reduction, chemical reduction and continuous-flow methods. These methods are often costly or produce by-products that pose increased risks to human health and the environment.

In recent years, green or environmentally friendly chemical methods have been developed to prepare nanoparticles using plant extracts. Green chemistry has the advantage of being safer, faster, environmentally friendly, and economical. However, the rise of green methods of preparing nanoparticles has also demonstrated that the activities and characteristics of the nanoparticles vary significantly, depending upon the detailed method of synthesis and specific plant extract used. Further, the therapeutic potential of plant extracts has been compromised due to the lack of controlled delivery of an effective dose to the desired target site.

Thus, nanoparticles synthesized using an environmentally friendly method solving the aforementioned problems are desired.

### SUMMARY

The present subject matter relates to a method of preparing selenium nanoparticles (Se NPs) using an edible mushroom extract. In an embodiment, the edible mushroom extract is prepared from a mushroom of the species Pleurotus floridanus. In an embodiment, the method includes combining the Pleurotus floridanus extract with a source of selenium to provide an extract mixture, maintaining the mixture at a pre-determined temperature until the selenium nanoparticles are formed, and separating the Pleurotus floridanus selenium nanoparticles from the extract mixture.

According to an embodiment, the present subject matter relates to a pharmaceutical composition comprising Se NPs and a pharmaceutically acceptable carrier. In an embodiment, the pharmaceutical composition includes an extract of Pleurotus floridanus.

According to an embodiment, the present subject matter relates to a method of inhibiting microbial growth in a subject comprising administering an effective amount of the pharmaceutical composition to a subject in need thereof. In an embodiment, the microbial growth includes growth Gram (+) bacteria, Gram (−) bacteria, and Candida.

These and other features of the present subject matter will become readily apparent upon further review of the following specification.

### BRIEF DESCRIPTION OF DRAWINGS

FIG. 1 is a UV-visible spectrum of Selenium (Se) salt and the Selenium (Se) nanoparticles after mushroom extract addition at pH 11 and their respective photographs.

FIG. 2 is a transmission electron microscopy (TEM) image of the selenium nanoparticles (Se NPs).

FIGS. 3A-3D show antimicrobial activity by zone of inhibited growth for FIG. 3A Bacillus cereus; FIG. 3B Staphylococcus aureus; FIG. 3C Escherichia coli; and FIG. 3D Klebsiella pneumoniae.

FIGS. 3E-3F show antimicrobial activity by zone of inhibited growth for FIG. 3E Candida parapsilosis; and FIG. 3F Candida albicans.

### DETAILED DESCRIPTION OF THE PREFERRED EMBODIMENTS

The following definitions are provided for the purpose of understanding the present subject matter and for construing the appended patent claims.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as a microbial infection.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In an embodiment, the present subject matter relates to a method of preparing selenium nanoparticles (NPs) using an edible mushroom extract. In an embodiment, the edible mushroom extract is prepared using a mushroom from the species Pleurotus floridanus. In an embodiment, the method includes combining the mushroom extract with a source of selenium to provide an extract mixture, and maintaining the mixture at a pre-determined temperature until the selenium nanoparticles are formed, and separating the Pleurotus floridanus selenium nanoparticles from the extract mixture. In an embodiment, the source of selenium can be sodium selenite (Na₂SeO₃). The selenium nanoparticles can be separated from the mixture in any suitable manner.

In an embodiment, the mixture can be maintained at a temperature ranging from about 25° C. to about 40° C., e.g., about 30° C., for about 12 hours to about 48 hours, e.g., about 24 hours, to provide the selenium nanoparticles (NPs). In an embodiment, a change of color of the extract mixture from colorless to red can indicate formation of the selenium nanoparticles (NPs). In an embodiment, the selenium nanoparticles can be separated from the mixture by centrifuging. In an embodiment, the extract mixture can be centrifuged at about 10,000 rpm to about 20,000 rpm, e.g., about 15,000 rpm.

In an embodiment, the edible mushroom in the extract is from the fungal species Pleurotus floridanus. In an embodiment, the selenium nanoparticles are spherical. Interestingly, the selenium nanoparticles (NPs) can be formed in the extract mixture without the addition of ascorbic acid.

The selenium nanoparticles can be effective against a variety of microorganisms, including Gram (+) bacteria, Gram (−) bacteria, and Candida. In an embodiment, the Gram (+) bacteria is selected from Bacillus cereus and Staphylococcus aureus. In an embodiment, the Gram (−) bacteria is selected from Escherichia coli and Klebsiella pneumoniae. In an embodiment, the Candida is selected from Candida parapsilosis and Candida albicans.

It is believed that the mushroom extract can have both reducing agents and stabilizing agents for reducing selenium ions to selenium zero-valent. In one embodiment, the mushroom extract can be an alcohol extract or a water extract, for example. Because Pleurotus floridanus has higher protein than expected from other mushroom species, this could help to prepare Se nanoparticles without use of a reducing agent or capping agent because of the interaction between the protein of Pleurotus floridanus and Selenium ions, and so, Pleurotus floridanus mushroom could be applied as a reducing, capping and stabilizing agent without using chemicals such as ascorbic acid or formation of composites.

In an embodiment, the Se NPs can have an average particle size ranging from about 5 nm to about 20 nm, or from about 9 nm to about 12 nm. In other embodiments, the Se NPs can have an average particle size of about 9 nm, about 10 nm, or about 12 nm.

An embodiment of the present subject matter is directed to a pharmaceutical composition comprising the Se NPs and a pharmaceutically acceptable carrier. In one embodiment, the pharmaceutical composition includes the mushroom extract, the Se NPs, and a pharmaceutically acceptable carrier.

An embodiment of the present subject matter is directed to a method of making a pharmaceutical composition including mixing the Se NPs and optionally, the mushroom extract, with a pharmaceutically acceptable carrier. For example, the method of making a pharmaceutical composition can include mixing the Se NPs and, optionally the mushroom extract, under sterile conditions with a pharmaceutically acceptable carrier with preservatives, buffers, and/or propellants to create the pharmaceutical composition.

To prepare the pharmaceutical composition, the Se-AgNPs, as the active ingredient, are intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by injection, inhalation or insufflation. The Se NPs can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the Se NPs or an amount effective to treat a disease, such as a microbial infection, may be determined initially from the Examples described herein and adjusted for specific targeted diseases using routine methods.

The Se NPs can have antibacterial and antifungal properties and can be particularly effective agents against Gram (+) bacteria, Gram (−) bacteria, and Candida. The Se NPs can be administered to a subject in need thereof. In an embodiment, the Se NPs can be administered to a subject in need thereof to inhibit bacterial growth. In an embodiment, the Se NPs can be administered to a subject in need thereof to inhibit fungal growth. In an embodiment, the Se NPs can be administered to a subject to inhibit the growth of Gram (+) bacteria selected from Bacillus cereus and Staphylococcus aureus. In an embodiment, the Se NPs can be administered to a subject to inhibit the growth of Gram (−) bacteria selected from Escherichia coli and Klebsiella pneumoniae. In an embodiment, the Se NPs can be administered to a subject to inhibit the growth of Candida parapsilosis and Candida albicans.

An embodiment of the present subject matter is directed to a method of inhibiting bacterial growth in a subject, comprising administering to a subject in need thereof a therapeutically effective amount of the pharmaceutical composition according to the present subject matter. An embodiment of the present subject matter is directed to a method of inhibiting fungal growth in a subject, comprising administering to a subject in need thereof a therapeutically effective amount of the pharmaceutical composition according to the present subject matter. In an embodiment, the fungal growth includes growth of Candida.

The Se NPs or pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### Example 1

### Synthesis of Selenium Nanoparticles

The selenium nanoparticles were synthesized by cutting edible mushrooms (Pleurotus floridanus) into small pieces and soaking the mushroom pieces in de-ionized water for 24 hours to provide a mushroom extract. The extract was separated from the solid mushroom pieces by filtration using Whatman filter paper No. 1. A source of selenium ions, sodium selenite (Na₂SeO₃), was added to the obtained filtrate to provide a selenium/mushroom extract mixture with zero valent selenium (Se) under room temperature conditions. The selenium/mushroom extract mixture was saved at 30° C. for 24 hours. A change in color of the mixture from colorless to red was the first indication of the formation of selenium nanoparticles (Se NPs). The obtained Se NPs mixture was centrifuged at 15,000 rpm for 15 minutes. The Se NPs material was purified by using pure H₂O to eliminate any adsorbed impurities.

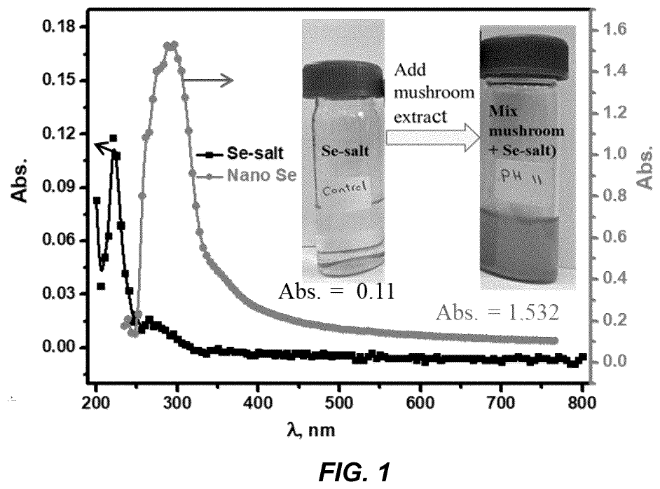

The optical scans of the Se NPs including the spectrophotometric scan of Se ions are shown in FIG. 1. A new peak was observed for the mixture of mushroom extract and selenium ions at λₘₐₓ=290 nm, indicating the successful formation of zero-valent selenium. As the reaction time increased, the absorbance values were enhanced to higher values. Therefore, a direct comparison between the Se concentration to form zero-valent Se by observation and absorbance values indicates the successful production of Se NPs using mushroom extract as a biocompatible reagent.

### Example 2

### Size and Morphology of Se NPs

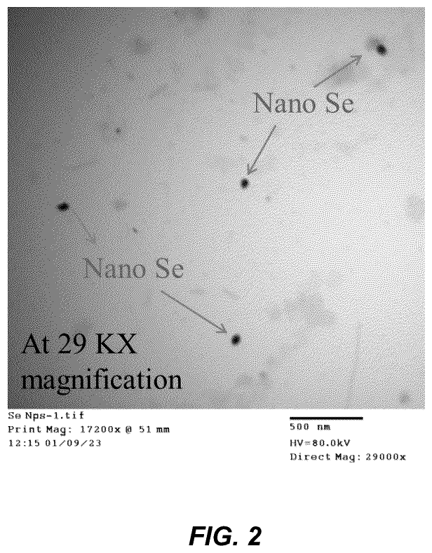

The Se NPs prepared using the edible mushroom as described above were characterized by TEM analysis to confirm the surface morphology. The TEM image was captured at a magnification of 29 KX as shown in FIG. 2. The morphology of the prepared zero-valent Se material was spherical with sizes ranging from 9 nm to 12 nm, which indicates the nano characteristics of the prepared zero-valent selenium. It is believed that these results are due to the use of mushroom extract (which has both a stabilizing agent and a reducing agent) in addition to the interaction between protein and selenium ions. The stability of the prepared selenium particles could be due to the chemical makeup of the mushrooms.

UV-visible spectroscopy of selenium (Se-salt and the formed selenium nanoparticles after mushroom extract addition at pH 11 and their photos are shown in FIG. 1. FIG. 2 shows the transmission electron microscopy (TEM) image of the synthesized selenium nanoparticles synthesized using mushroom extract.

### Example 3

Anti-Fungal Activity of AH—Ag-Nps and Amaranthus hybridus Extract (AH)

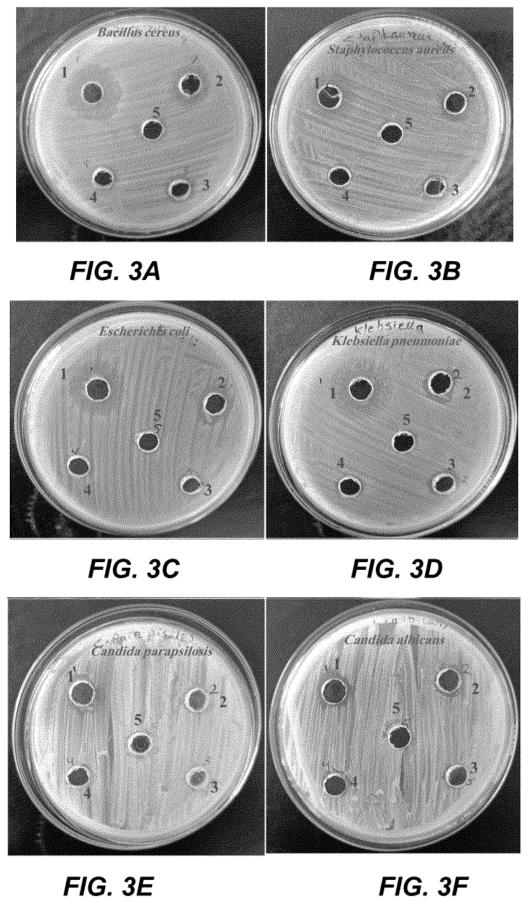

The antibacterial activities of the introduced Se NPs were evaluated against Gram (+) bacteria, including Bacillus cereus and Staphylococcus aureus, Gram (−) bacteria, including Escherichia coli and Klebsiella pneumoniae, and Candida, including, Candida parapsilosis and Candida albicans, using the agar well diffusion method. The results of the tests are shown in Table 1, below. As shown in Table 1 and FIGS. 3A-3F, the inhibition zone for Bacillus cereus and Staphylococcus aureus was found to be at 20 mm and 18 mm, respectively. This inhibition zone was influenced by the concentration of the Se NPs. As the concentration decreased, the inhibition zone decreased at all studied concentrations. For Se NPs evaluated against Escherichia coli and Klebsiella pneumoniae, the inhibition zone was found to be at 22 mm and 25 mm, respectively which indicates the higher impact of Se NPs over the Gram (−) bacteria than the Gram (+) ones. Additionally, for Se NPs evaluated against Candida parapsilosis and Candida albicans, the inhibition zone was found to be at 13 mm and 12 mm, respectively. Therefore, the Se NPs have a considerable impact over the tested microorganisms, indicating their potential utility as an antimicrobial agent for commercial biomedical applications.

TABLE 1
Antimicrobial activity of the prepared Se NPs

| | | | | | Klebsiella | | |
|---|---|---|---|---|---|---|---|
| | | B. cereus | S. aureus | E. coli | pneumoniae | | |
| | Conc. | (+ve) | (+ve) | (−ve) | (−ve) | C. parapsilosis | C. albicans |
| No. | μM | IZD ^{a} | IZD ^{a} | IZD ^{a} | IZD ^{a} | IZD ^{a} | IZD ^{a} |
| 1 | 100 | 20 | 18 | 22 | 25 | 13 | 12 |
| 2 | 80 | 15 | 14 | 18 | 19 | 10 | 10 |
| 3 | 60 | 13 | 12 | 13 | 14 | 0 | 0 |
| 4 | 40 | 10 | 10 | 10 | 9 | 0 | 0 |
| 5 | 20 | 0 | 0 | 0 | 0 | 0 | 0 |

^{a} IZD is the inhibition zone diameter by mm using agar diffusion assay.

It is to be understood that the nanoparticles described herein are not limited to the specific embodiments described above, but encompass any and all embodiments within the scope of the generic language of the following claims enabled by the embodiments described herein, or otherwise shown in the drawings or described above in terms sufficient to enable one of ordinary skill in the art to make and use the claimed subject matter.

1. A method of synthesizing selenium nanoparticles, consisting of:
mixing an extract of Pleurotus floridanus with sodium selenite Na₂SeO₃ to provide an extract mixture;
maintaining the extract mixture for a predetermined period of time and at a predetermined temperature to provide Pleurotus floridanus selenium nanoparticles; and
separating the Pleurotus floridanus selenium nanoparticles from the extract mixture.

2. The method of claim 1, wherein the predetermined temperature ranges from about 25° C. to about 40° C.

3. The method of claim 1, wherein the predetermined time ranges from about 12 hours to about 48 hours.

4. Selenium nanoparticles prepared according to the method of claim 1.

5. The selenium nanoparticles of claim 4, having an average particle size ranging from about 5 nm to about 20 nm.

6. A pharmaceutical composition, comprising the Pleurotus floridanus silver nanoparticles of claim 4 and a pharmaceutically acceptable carrier.

7. The pharmaceutical composition of claim 6, further comprising an extract of Pleurotus floridanus.

8.-14. (canceled)

15. A method of synthesizing selenium nanoparticles, consisting of:
mixing an extract of Pleurotus floridanus with sodium selenite Na₂SeO₃ to provide an extract mixture;
maintaining the extract mixture for a period of time ranging from about 12 hours to about 48 hours and at a temperature ranging from about 25° C. to about 40° C. to provide selenium nanoparticles; and
separating the Pleurotus floridanus selenium nanoparticles from the extract mixture.

16. Selenium nanoparticles prepared according to the method of claim 15.

17. The selenium nanoparticles of claim 16, wherein a size of the selenium nanoparticles ranges from about 5 nm to about 20 nm.
